(19) United States
(12) Patent Application Publication
(10) Pub. No.: US 2006/0035879 A1
(43) Pub. Date: Feb. 16, 2006

(54) Organic Compounds

(76) Inventor: Margaret Forney Prescott, Millburn, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/534,790

(22) PCT Filed: Nov. 14, 2003
(86) PCT No.: PCT/EP03/12737

§ 371 Date: Jul. 20, 2005

Related U.S. Application Data
(60) Provisional application No. 60/426,809, filed on Nov. 15, 2002.

(51) Int. Cl. A61K 31/33; A61M 31/00

(52) U.S. Cl. 514/183; 424/423; 604/500

(57) ABSTRACT

A drug delivery device or system comprising: a) a medical device, e.g. a coated stent or stent-graft, adapted for local application or administration in hollow tubes; and in conjunction therewith, b) a therapeutic dosage of an anti-inflammatory ascomycin derivative, such as pimecrolimus, e.g. affixed to the medical device, and use thereof in the preparation of a medicament for the prevention and treatment of inflammatory complications following vascular injury, and method of treatment therewith.

The invention relates to organic compounds, more particularly to drug delivery systems for the prevention and treatment of inflammatory or proliferative diseases, particularly vascular inflammatory and/or hyperproliferative and/or matrix degradative diseases.

Many patients suffer from circulatory diseases caused by a progressive blockage of the blood vessels that perfuse major organs such as heart, liver, kidney and brain. Severe blockage of blood vessels often leads to e.g. ischemic injury, hypertension, stroke or myocardial infarction. Atherosclerotic lesions which limit or obstruct coronary or peripheral blood flow are the major cause of ischemic disease-related morbidity and mortality, including coronary heart disease, stroke, aneurysm and peripheral claudication.

To stop the disease process and prevent the more advanced disease states in which the cardiac muscle or other organs or vessels themselves are compromised, medical revascularization and/or repair procedures such as percutaneous transluminal coronary angioplasty (PCTA), percutaneous transluminal angioplasty (PTA), stenting, atherectomy, or other types of catheter-based revascularization/ local drug delivery techniques at the site of the disease, either applied via the vessel lumen or applied via the external/adventitial aspect of the vessel, such as those grafts or other devices used to repair aneurysm, as well as by-pass grafting are used. Ultrasound or other techniques resulting in activation or delivery of drug-containing microbubbles or liposomes or other vehicles that carry drug for local delivery is also used as a mechanism of local drug delivery during revascularization or as a mechanism of revascularization. In addition to the proliferative narrowing, occlusion or constrictive remodeling seen in native arteries after revascularization or within by-pass grafts, at sites of anastomoses in transplantation or aneurysm, or in veins post-injury or thrombosis, there is also a pathological outward remodeling (or ballooning out) that occurs at sites of aneurysm that can still occur despite surgical or endolumenal attempts to repair and stabilize these sites. Stabilization/repair of aneurysm using endovascular devices such as stents or sleeves or other endovascular devices and/or other local delivery methods such as adventitial wrapping can also be performed together with local delivery/elution of drug to enhance stabilization of the vessel wall or prevent progression of the aneurysm to adjacent sections of vessel. Thus revascularization procedures such as angioplasty and/or stenting and/or other types of catheter-based local delivery as well as endovascular devices and adventitial wraps are used in a wide variety of vascular pathologic conditions and can all be used as platforms to deliver drug to the vessel wall to prevent re-closure and/or prevent progression of aneurysm and/or to otherwise repair or stabilize the vessel.

Re-narrowing, e.g. of an atherosclerotic coronary artery after various revascularization procedures or exacerbated aneurysm (outward dilation), e.g. of the aorta after various endovascular aneurysm repair, occurs in about 10 to 80% of patients undergoing these treatments, depending on the procedure used as well as the arterial or venous site. Besides opening an artery obstructed by atherosclerosis, revascularization in general, but especially revascularization using a stent, injures endothelial cells and smooth muscle cells within the vessel wall, thus initiating or exacerbating a thrombotic and inflammatory response that is often followed by a proliferative response or sometimes a response in which the vessel wall is degraded. Cell-derived growth factors such as platelet derived growth factors, endothelial-derived growth factors, smooth muscle-derived growth factors (e.g. PDGF, tissue factor, FGF), as well as cytokines, chemokines, lymphokines or proteases released from endothelial cells, infiltrating macrophages, lymphocytes or leukocytes, or released from the smooth muscle cells themselves, provoke proliferative and migratory responses in the smooth muscle cells as well as additional inflammatory events, or provoke matrix deposition or its reverse, matrix degradation, as well as neovascularization within the vessel wall. Effects on the vascular smooth muscle cells usually begins within one to two days post-revascularization and/or device placement and, depending on the revascularization procedure or endovascular device used, continues for days, weeks, or even months.

Cells within the original atherosclerotic lesion or aneurysm as well as inflammatory cells that have accumulated at the site of injury and stenting or grafting, as well as smooth muscle cells within the media migrate, proliferate and/or secrete significant amounts of extracellular matrix proteins and/or proteases. In an artery or vein, proliferation, migration and extracellular matrix synthesis continue until the damaged endothelial layer is repaired, at which time proliferation may slow within the intima. The newly formed tissue following stenting is named neointima, intimal thickening or restenotic lesion, and usually results in narrowing of the vessel lumen. Further lumen narrowing may take place due to constructive remodeling, e.g. vascular remodeling, leading to further loss of lumen size. In an aneurysm, inflammatory cells such as lymphocytes and monocytes accumulate following endovascular aneurysm repair and both inflammatory cells and smooth muscle cells secrete proteases that further degrade the matrix.

However, restenosis remains a major problem in percutaneous coronary intervention, and lack of aneurysm stabilization remains a major problem in endovascular stent/graft placement for aneurysm, requiring patients to undergo repeated procedures and surgery. Restenosis is the result of the formation of neointima, a composition of smooth muscle-like cells in a collagen matrix. Aneurysm progression is a result of vessel wall expansion, usually due to inflammatory cell accumulation, matrix degradation and smooth muscle cell apoptosis.

A major category of interventional devices called stents has been introduced with the aim of reducing the restenosis rate of balloon angioplasty and reducing the complications of aortic aneurysm surgery.

Clinical studies have shown a reduction in the restenosis rates as compared with angioplasty and reduction of aneurysm progression using endovascular aneurysm repair compared with surgery using stents. The purpose of stenting for both revascularization and aneurysm is to maintain the arterial lumen by a scaffolding process that provides radial support. Stents, usually made of stainless steel or of a synthetic material, are placed in the artery either by a self-expanding mechanism or using balloon expansion or are placed in the aorta as part of a graft. Stenting results in the largest lumen possible and expands the artery to the greatest degree possible. Stenting also provides a protective frame to support fragile vessels that have had a pathologic dissection due to the revascularization procedures or due to aneurysm. It has been demonstrated that the implantation of stents as part of the standard angioplasty procedure improves the acute results of percutaneous coronary revascularization, but in-stent restenosis, as well as stenosis proximal and distal to the stent and the inaccessibility of the lesion site for surgical revasculation limits the long-term success of using stents. The absolute number of in-stent restenotic lesions is increasing with the increasing number of stenting procedures, with the complexity of culprit lesion stented as well as with stenting of ever-smaller sized arteries. Neointima proliferation/growth occurs principally within the stented area or proximal or distal to the stented area within 6 months after stent implantation. Neointima is an accumulation of smooth muscle cells within a proteo-glycan matrix that narrows the previously enlarged lumen. It has likewise been demonstrated that use of endovascular devices to repair aneurysm improves the results of aneurysm repair.

Attempts have been made to orally treat restenosis following stenting or aneurysm following endovascular device placement with various pharmaceutically active agents, however, these attempts have usually failed.

A recent development in the stent device area is the use of stents that release or elute pharmacological agents having antiproliferative and/or antiinflammatory activity.

However, there is a need for farther effective approaches for treatments and the use of drug delivery systems for preventing and treating intimal thickening or restenosis that occurs after injury due to stenting, e.g. vascular injury, including e.g. surgical injury, e.g. revascularization-induced injury, e.g. anastomotic sites for heart or other sites of organ transplantation, or for preventing and treating aneurysm expansion that occurs after stenting or grafting e.g. following endovascular aneurysm repair.

A further application of stenting is emerging, namely for vulnerable plaque or aneurysm stabilization. Vulnerable plaques are those atherosclerotic lesions that are prone to rupture or ulceration, resulting in thrombosis and thus producing unstable angina, myocardial infarction or sudden death. Such plaques are often not flow-limiting, e.g. they do not cause stenosis that closes the vessel by more than 50%. However, vulnerable plaques that are not flow-limiting, e.g. in which stenosis is less than 50%, may be stented to stabilize the vulnerable plaque so that it does not rupture, as contrasted with opening up a stenotic vessel to allow more blood to flow through as is done via re-vascularization. Aneurysms are outward dilation of a vessel, usually the aorta, that can rupture and cause hemorrhage. Such aneurysms may be stented or repaired with devices containing elements of both stents and grafts via endovascular techniques.

Ascomycin derivatives have anti-inflammatory and/or immunosuppressant properties and may be used e.g. for immunosuppression or in the treatment of inflammatory skin diseases.

Surprisingly, it has now been found that anti-inflammatory ascomycin derivatives, especially pimecrolimus, optionally administered together with other active agents, e.g. antiproliferative compounds or protease inhibitors, have beneficial effects when locally applied to the lesions sites in vascular disease, including stenoses or aneurysm or vulnerable plaques, or when used e.g. systemically in conjunction with interventional devices locally applied to the lesion sites in vascular disease.

Hence, the invention relates to a method for preventing and treating inflammatory complications following vascular injury, in particular intimal thickening or restenosis that occurs after vascular injury, including e.g. surgical injury, e.g. revascularization-induced injury, e.g. also in heart or other grafts, and relates to a method for preventing or treating aneurysm progression or rupture following endovascular stent grafting for aneurysm, and involves administering a therapeutically effective amount of an anti-inflammatory ascomycin derivative to a mammal, e.g. a patient, in need thereof.

In addition, anti-inflammatory ascomycin derivatives may also advantageously inhibit and possibly even reverse angiogenesis associated with diseases or pathological conditions in mammals. Thus treatment therewith of patients with atherosclerotic plaques or aneurysm may advantageously result in stabilisation of atherosclerotic plaques and of sites of aneurysm, and thus in inhibition of angiogenesis associated with plaque instability and rupture or aneurysm expansion which can result in thrombosis and the like, thereby decreasing the risk of thrombosis, unstable angina, myocardial infarction, sudden death, stroke, and aneurysm expansion and hemorrhage; preferably in conjunction with a medical device adapted for local application or administration in hollow tubes, such as a stent.

The invention particularly concerns drug delivery devices or systems comprising:
a) a medical device, e.g. a catheter-based delivery device or an intraluminal device, especially a coated stent or stent-graft, adapted for local application or administration in hollow tubes; and, in conjunction therewith,
b) a therapeutic dosage of an anti-inflammatory ascomycin derivative, optionally together with a therapeutic dosage of one or more other active ingredients, preferably each being affixed to the medical device in a way allowing drug release;
   hereinafter briefly named “the device of the invention”.

A device of the invention preferably comprises an endovascular device, e.g. a stent or stent-graft, especially a coated stent.

The invention also concerns the use of an anti-inflammatory ascomycin derivative in the preparation of a medicament for the prevention and treatment of inflammatory complications following vascular injury, such as:
the prevention or treatment, e.g. systemically, preferably locally, of vascular inflammation or smooth muscle cell proliferation and migration, or aneurysm expansion in hollow tubes, or increased extracellular matrix degradation and erosion in hollow tubes, or increased inflammatory cell infiltration, or increased cell proliferation or decreased apoptosis, or increased matrix deposition or degradation, or increased positive, aneurysmal remodeling (aneurysm dilation) following device placement; or
the treatment of intimal thickening or aneurysm expansion in vessel walls; or
stabilising atherosclerotic plaques, or stabilising sites of aneurysm; or
stabilising or reducing aneurysm dilation at the site of aneurism in e.g. the aorta or other vessels following device placement;
   preferably in conjunction with a medical device as defined under a) above.

An “ascomycin derivative” is to be understood herein as being an antagonist, agonist or analogue of the parent compound ascomycin which retains the basic structure and modulates at least one of the biological, for example immunological properties of the parent compound.

An “anti-inflammatory ascomycin derivative” is defined herein as being an ascomycin derivative that exhibits pronounced anti-inflammatory activity in e.g. animal models of allergic contact dermatitis but has only low potency in suppressing systemic immune response, namely, which has a minimum effective dose (MED) of up to a concentration of about 0.04% w/v in the murine model of allergic contact dermatitis upon topical administration, while its potency is at least 10 times lower than for tacrolimus (MED 14 mg/kg) in the rat model of allogeneic kidney transplantation upon oral administration (Meingassner, J. G. et al., Br. J. Dermatol. 137 [1997] 568-579; Stuetz, A. Seminars in Cutaneous Medicine and Surgery 20 [2001] 233-241). Such compounds are preferably lipophilic.

An anti-inflammatory ascomycin derivative may be in free form or, where such forms exist, in pharmaceutically acceptable salt form.

Suitable anti-inflammatory ascomycin derivatives are e.g.:
(32-desoxy-32-epi-N1-tetrazolyl)ascomycin (ABT-281) (J.Invest.Dermatol. 12 [1999] 729-738, on page 730, FIG. 1);
{1E-(1R,3R,4R)]1R,4S,5R,6S,9R,10E,13S,15S,16R,17S,19S,20S}-9-ethyl-6,16,20-trihydroxy-4-[2-(4-hydroxy-3-methoxycyclohexyl)-1-methylvinyl]-15,17-dimethoxy-5,11,13,19-tetramethyl-3-oxa-22-azatricyclo[18.6.1.0(1,22)]heptacos-10-ene-2,8,21,27-tetraone (Examples 6d and 71 in EP 569337), hereinafter referred to as “ASD 732”;
{1R,5Z,9S,12S-[1E-(2R,3R,4R)],13R,14S,17R,18E,21S,23S,24R,25S,27R}-17-ethyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0(4,9)]octacos-5,18-diene-2,3,10,16-tetraone (Example 8 in EP 626385), hereinafter referred to as “5,6-dehydroascomycin”; and
33-epichloro-33-desoxyascomycin (ASM 981), i.e. {[1E-(1R,3R,4S)]1R,9S,12S,13R,14S,17R,18E,21S,23S,24R,25S,27R}-12-[2-(4-chloro-3-methoxycyclohexyl)-1-methylvinyl]-17-ethyl-1,14-dihydroxy-23,25-dimethoxy-13,19,21,27-tetramethyl-11,28,dioxa-4-azatricyclo [22.3.1.0(4,9)]octacos-18-ene-2,3,10,16-tetraone,
   (Example 66a in EP 427680); hereinafter referred to as pimecrolimus (INN) (Elidel^{R}).

Particularly preferred is pimecrolimus; it is in free form unless specified otherwise herein.

The anti-inflammatory ascomycin derivatives may be prepared and administered in conventional manner.

The structure of the active ingredients identified by code numbers, generic or trade names may be taken from the standard compendium “The Merck Index” or from computer databases, e.g. Patents International (e.g. IMS World Publications). The corresponding content thereof is hereby incorporated by reference. Any person skilled in the art is fully enabled to identify the active ingredients and, based on these references, likewise enabled to manufacture and test the pharmaceutical indications and properties in standard test models, both in vitro and in vivo.

The anti-inflammatory ascomycin derivatives may be applied as the sole active ingredient or together with at least one other pharmacologically active agent, e.g. with:
an immunosuppressive agent, e.g. a mitogen-activated kinase modulator or inhibitor, such as e.g. a rapamycin, e.g. sirolimus or everolimus;
an EDG-receptor agonist, e.g. FTY720;
another anti-inflammatory agent, e.g. a steroid, e.g. a corticosteroid, e.g. dexamethasone or prednisone;
a NSAID, e.g. a cyclooxygenase inhibitor, e.g. a COX-2 inhibitor, e.g. celecoxib, rofecoxib, etoricoxib or valdecoxib;
an anti-thrombotic or anti-coagulant agent, e.g. heparin or a IIb/IIIa inhibitor;
an antiproliferative agent, e.g. a microtubule stabilizing or destabilizing agent, including but not limited to taxanes, e.g. taxol, paclitaxel or docetaxel;
vinca alkaloids, e.g. vinblastine, especially vinblastine sulfate, vincristine, especially vincristine sulfate and vinorelbine;
discodermolides or epothilones or a derivative thereof, e.g. epothilone B or a derivative thereof;
staurosporin and related small molecules, e.g. UCN-01, BAY 43-9006, Bryostatin 1, Perifosine, Limofosine, midostaurin, RO318220, RO320432, GO 6976, Isis 3521, LY333531, LY379196, SU5416, SU6668 or AG1296;
a compound or antibody which inhibits the PDGF receptor tyrosine kinase or a compound which binds to PDGF or reduces expression of the PDGF receptor, e.g. STI571, CT52923, RP-1776, GFB-111 or a pyrrolo[3,4-c]-beta-carboline-dione;
compounds affecting GRB2, e.g. IMC-C225;
statins, e.g. having HMG-CoA reductase inhibiting activity, e.g. fluvastatin, lovastatin, simvastatin, pravastatin, atorvastatin, cerivastatin, pitavastatin, rosuvastatin or nivastatin;
a compound, protein, growth factor or compound stimulating growth factor production that will enhance endothelial re-growth of the luminal endothelium, e.g. FGF, IGF, a matrix metalloproteinase inhibitor, e.g. batimistat, marimistat, trocade, CGS 27023, RS 130830 or AG3340;
a modulator (i.e. antagonist or agonist) of kinases, e.g. JNK, ERK1/2, MAPK or STAT;
an isosorbide compound; or
an NF-κB inhibitor.

The invention thus may also be effected e.g. by local administration or delivery of an anti-inflammatory ascomycin derivative together with at least one other pharmacologically active agent, e.g. an agent as defined above.

Further, the invention concerns a method of treatment of inflammatory complications following vascular injury, such as for:
preventing or treating vascular inflammation or smooth muscle cell proliferation and migration, or aneurysm expansion in hollow tubes, or increased extracellular matrix degradation and erosion in hollow tubes such as arteries or veins, or increased inflammatory cell infiltration, or increased cell proliferation or decreased apoptosis, or increased matrix deposition or degradation, or increased positive, aneurysmal remodeling (aneurysm dilation) following device placement in a mammal in need thereof, comprising systemic or, preferably, local administration of a therapeutically effective amount of an anti-inflammatory ascomycin derivative, e.g. following device placement;
treating intimal thickening or aneurysm expansion in vessel walls in a mammal in need thereof, comprising controlled delivery from a catheter-based or intraluminal medical device of a therapeutically effective amount of an anti-inflammatory ascomycin derivative, optionally together with one or more other active ingredients, e.g. as disclosed above; preferably in conjunction with a medical device as defined under a) above;
stabilising atherosclerotic plaques or stabilising sites of aneurysm, or stabilising or reducing aneurysm dilation at the site of aneurysm e.g. in the aorta or other vessels following device placement in a mammal in need thereof, comprising systemic or, preferably, local administration of a therapeutically effective amount of an anti-inflammatory ascomycin derivative, optionally together with one or more other active ingredients, e.g. as disclosed above; preferably in conjunction with a medical device as defined under a) above.

The underlying condition beneficially affected is e.g. stenosis; restenosis, e.g. following revascularization or neovascularization; vascular inflammation; thrombosis; unstable angina; myocardial infarction; heart failure; ischaemia; sudden death; stroke; and/or aneurysm rupture. Preferably the anti-inflammatory ascomycin derivative is administered from stents or from a coating applied to stents, or in conjunction with stents.

A device of the invention can be used to reduce stenosis or restenosis or aneurysm dilation as an adjunct to revascularization, by-pass or grafting procedures performed in any vascular location including coronary arteries, carotid arteries, renal arteries, peripheral arteries, cerebral arteries, aorta or any other arterial or venous location, to reduce anastomic stenosis such as in the case of arterial anastomoses in transplant, to reduce aneurysm dilation and rupture with or without endovascular devices such as stent-grafts, or in conjunction with any other heart or transplantation procedures, or congenital vascular interventions.

“Treatment” herein means prophylactic as well as curative treatment.

“Hollow tube” means any physiological hollow tube that has the function of transporting a gas or liquid, preferably a liquid, and most preferably blood, for example a vessel, vein, artery, etc., and that can be affected by atherosclerosis, thrombosis, restenosis, aneurysm and/or vascular inflammation.

“Together with” should be understood to apply to either temporal proximity, as with e.g. more or less simultaneous administration, or to physical proximity, or both.

An anti-inflammatory ascomycin derivative is referred to hereinafter as “drug”. The other active ingredients which may be used together with the anti-inflammatory ascomycin derivative, e.g. as disclosed above, are referred to hereinafter collectively as “adjunct”.

“Drug(s)” means drug or drug plus adjunct.

“Local” administration preferably takes place at or near the vascular lesions sites. Local drug(s) administration may be e.g. by one or more of the following routes: via catheter or other intravascular delivery system; intranasally; intrabronchially; interperitoneally, or via the eosophage. Hollow tubes include circulatory system vessels, such as blood vessels (arteries or veins), tissue lumen, lymphatic pathways, digestive tract including alimentary canal, respiratory tract, excretory system tubes, reproductive system tubes and ducts, body cavity tubes, etc. Local administration or application of the drug(s) affords concentrated delivery of said drug(s), achieving tissue levels in target tissues not otherwise obtainable through other administration route.

Means for local drug(s) application or administration (delivery) to hollow tubes can be by physical delivery of the drug(s) either internally or externally to the hollow tube. Local drug(s) delivery includes catheter-based delivery devices, local injection devices or systems, or intraluminal or indwelling devices adapted for local application or administration in hollow tubes. Such devices or systems include, but are not be limited to, stents, coated stents, endoluminal sleeves, stent-grafts, liposomes, controlled release matrices, polymeric or biological endoluminal paving or other endovascular devices, adventitial wraps, embolic delivery particles, cell targeting such as affinity-based delivery, internal patches around the hollow tube, external patches around the hollow tube, hollow tube cuff, external paving, external stent sleeves, and the like, as described in Eccleston et al. Interventional Cardiology Monitor 1 [1995] 33-40-41; Slepian Intervente. Cardiol. 1 [1996] 103-116; and Regar et al., “Stent development and local drug delivery”, Br. Med. Bull. 59 [2001] 227-48, which disclosures are herein incorporated by reference.

Drug delivery may optionally take place from the outside of the vessel to the inside of the vessel, whereby the drug is impregnated in devices applied to the external surface of an artery or vein.

Systemic administration of drug(s) takes place in conventional manner, e.g. orally.

“Biocompatible” is meant herein as a material which elicits no or only minimal negative tissue reaction, including e.g. thrombus formation and/or inflammation.

Delivery or application of the drug(s) can occur using e.g. stents or sleeves or sheathes. An intraluminal stent composed of, or coated with, a polymer or other biocompatible material, e.g. porous ceramic, e.g. nanoporous ceramic, into which the drug(s) has been impregnated or incorporated can be used. Such stents can be biodegradable or can be made of metal or alloy, e.g. Ni and Ti, or another stable substance when intented for permanent use. The drug(s) may also be entrapped into the metal of the stent or graft body which has been modified to contain micropores or channels. Lumenal and/or ablumenal coating or external sleeve made of polymer or other biocompatible materials, e.g. as disclosed above, that contain the drug(s) can also be used for local delivery.

Stents are commonly used as a tubular structure left inside the lumen of a duct or vessel to relieve an obstruction. They may be inserted into the duct lumen or lumen in a non-expanded form and are then expanded autonomously (self-expanding stents) or with the aid of a second device in situ, e.g. a catheter-mounted angioplasty balloon which is inflated within the stenosed vessel or body passageway in order to shear and disrupt the obstructions associated with the wall components of the vessel and to obtain an enlarged lumen.

Stent coating may be effected in conventional manner, e.g. by spraying drug onto the stent, by affixing it onto a semi-synthetic polymer, or by affixing it onto a biological polymer.

For example, the drug(s) may be incorporated into or affixed to the stent in a number of ways and utilizing any biocompatible materials; it may be incorporated into e.g. a polymer or a polymeric matrix and sprayed onto the outer surface of the stent. A mixture of the drug(s) and the polymeric material may be prepared in a solvent or a mixture of solvents and applied to the surface of the stents also by dip-coating, brush coating and/or dip/spin coating, the solvent(s) being allowed to evaporate to leave a film with entrapped drug(s). In the case of stents where the drug(s) is delivered from micropores, struts or channels, a solution of a polymer may additionally be applied as an outlayer to control the drug(s) release; alternatively, the drug(s) may be comprised in the micropores, struts or channels and the adjunct may be incorporated in the outlayer, or vice versa. The drug may also be affixed in an inner layer of the stent and the adjunct in an outer layer, or vice versa. The drug(s) may also be attached by a covalent bond, e.g. esters, amides or anhydrides, to the stent surface, involving chemical derivatization. The drug(s) may also be incorporated into a biocompatible porous ceramic coating, e.g. a nanoporous ceramic coating.

When drug is administered systemically, an adjunct may be administered either locally as described above, or systemically as well.

Examples of polymeric materials include biocompatible degradable or erodible materials, e.g. lactone-based polyesters or copolyesters, e.g. polylactide; polylactide-glycolide; polycaprolactone-glycolide; polyorthoesters; polyanhydrides; polyaminoacids; polysaccharides; polyphosphazenes; poly(ether-ester) copolymers, e.g. PEO-PLLA, or mixtures thereof; and biocompatible non-degrading materials, e.g. polydimethylsiloxane; poly(ethylene-vinylacetate); acrylate based polymers or coplymers, e.g. polybutylmethacrylate, poly(hydroxyethylmethyl-methacrylate); polyvinyl pyrrolidinone; fluorinated polymers such as polytetrafluoethylene; and cellulose esters.

When a polymeric matrix is used, it may comprise 2 layers, e.g. a base layer in which the drug(s) is incorporated, e.g. ethylene-co-vinylacetate and polybutylmethacrylate, and a top coat, e.g. polybutylmethacrylate, which is drug(s)-free and acts as a diffusion-control of the drug(s). Alternatively, the drug may be comprised in the base layer and an adjunct may be incorporated in the outlayer, or vice versa. Total thickness of the polymeric matrix may be from about 1 to about 20 μm or greater.

The drug(s) may elute passively, actively or under activation, e.g. light-activation.

The drug(s) elutes from the polymeric material or the stent over time and enters the surrounding tissue, e.g. for up to about 1 month to 1 year. Local delivery allows for high concentration of the drug(s) at the disease site with low concentration of circulating compound. The amount of drug(s) used for local delivery applications will vary depending on the compounds used, the condition to be treated and the desired effect. For purposes of the invention, a therapeutically effective amount will be administered. By therapeutically effective amount is meant an amount sufficient to inhibit cellular proliferation and resulting in the prevention and treatment of the disease state. Specifically, for the prevention or treatment of restenosis e.g. after revascularization, local delivery may require less compound than systemic administration.

The utility of the drug(s) may be demonstrated in animal test methods as well as in clinic, for example in accordance with conventional methods and/or the methods described herein.

The following Examples illustrate the invention and are not limitative. All temperatures are in degrees Centigrade. The abbreviations used have the following meanings:
ANOVA=analysis of variance
BrDU=bromodeoxyuridine
EEL=external elastic lamina
EL=internal elastic lamina
MW=molecular weight
P=probability
PBS=phosphate buffer solution
PGDF=platelet-derived growth factor
PEG=polyethyleneglycol
SEM=standard error from the mean

### EXAMPLE 1

### Effects of Orally Delivered vs Locally Delivered Drug on Inflammatory Cell Infiltration at 1 Day, or Early Neointimal Lesion Formation at 9 Days, Versus Late Neointimal Lesion Formation at 21 Days in the Rat Carotid Artery Balloon Injury Model

Numerous compounds have been shown to inhibit intimal lesion formation at 2 weeks in the rat ballooned carotid model, while only few compounds prove effective at 4 weeks. The compounds used according to the present invention are tested in the following rat model:

Rats are dosed orally with placebo or an anti-inflammatory ascomycin derivative. Daily dosing starts 0 to 5 days prior to surgery and continues up to an additional 28 days. Rat carotid arteries are balloon injured as described by Clowes et al., Lab. Invest. 49 (1983) 208-215. Quantitation of vascular inflammatory cell number is performed using cell flow cytometry [Hay C. et al., Arterioscler. Thromb. Vasc. Biol. 21 (2001) 1948-1954]. In studies determining lesion size, BrDU is administered for 24 hours prior to sacrifice. Sacrifice is performed at 1, 9 or 21 days post-balloon injury. Carotid arteries are removed and processed for flow cytometry or histologic and morphometric evaluation.

In this assay, the ability of pimecrolimus can be demonstrated to significantly reduce CD45-positive leukocyte infiltration into the vessel wall and adventitia at 1 day and to significantly reduce neointimal lesion formation following balloon injury at 9 and 12 days. Furthermore, when pimecrolimus is administered locally to the adventitia adjacent to the ballooned carotid (via a cather implanted into the adventitia that is connected to an Alzet minipump containing pimecrolimus suspended in vehicle), there is potent inhibition of infiltration of CD45⁺ leukocytes at day 1 and both early (9 days post-ballooning) and late (21-28 days post-ballooning) neointimal lesions, as well as potent inhibition of constrictive remodeling.

### EXAMPLE 2

### Inhibition of In-Stent Restenosis and Proximal and Distal Lesion Development at 28 Days in the Rabbit Iliac Stent Model

A combined angioplasty and stenting procedure is performed in New Zealand White rabbit iliac arteries. Iliac artery balloon injury is performed by inflating a 3.0×9.0 mm angioplasty balloon in the mid-portion of the artery followed by “pull-back” of the catheter for 1 balloon length. Balloon injury is repeated 2 times, and a 3.0×12 mm stent is deployed at 6 atm for 30 seconds in the iliac artery. Balloon injury and stent placement is then performed on the contralateral iliac artery in the same manner. A post-stent deployment angiogram is performed. All animals receive oral aspirin 40 mg/day daily as anti-platelet therapy and are fed standard low-cholesterol rabbit chow. Twenty-eight days after stenting, animals are anesthetized and euthanized and the arterial tree is perfused at 100 mmHg with lactated Ringer's solution for several minutes, then perfused with 10% formalin at 100 mmHg for 15 minutes.

The vascular section between the distal aorta and the proximal femoral arteries is excised and cleaned of periadventitial tissue. Three sections of artery are sampled: the stented section, the artery 5 mm immediately proximal to the stent and the artery 5 mm immediately distal to the stent is embedded in plastic. Sections are taken from the proximal, middle, and distal portions of each stent. Serial sections are also taken of the first 2 mm proximal and distal to the stent. Sections are stained with hematoxylin-eosin and Movat pentachrome stains. Other sections are stained with species-specific antibodies to allow immunocytochemical identification of macrophages. A non-specific isotype antibody is used as a negative control. Computerized planimetry is performed to determine the area of the IEL, EEL and lumen. The neointima and neointimal thickness is measured both at and between the stent struts. The vessel area is measured as the area within the EEL. Cells staining positively as macrophages are counted in the sections taken from the stented area of artery. Data are expressed as mean ±SEM. Statistical analysis of the histologic data is accomplished using ANOVA due to the fact that two stented arteries are measured per animal with a mean generated per animal. A “P” value of <0.05 is considered statistically significant.

Pimecrolimus is administered orally by gavage at an initial dose one day prior to stenting, then dosed at 50% of the initial dose from the day of stenting until day 27 post-stenting. In this model a marked reduction in the extent of restenotic lesion formation in the presence of pimecrolimus can be shown, whereas there is extensive neointimal formation in placebo-treated animals at 28 days, with the lesions consisting of abundant smooth muscle cells in proteoglycan/collagen matrix and apparent full endothelial healing. In addition, lesion formation in the portions of artery immediately proximal and immediately distal to the stent is also inhibited in the animals treated with pimecrolimus compared to those treated with placebo. Furthermore, the number of inflammatory cells, especially those in the area surrounding the stent struts, is significantly reduced in pimecrolimus samples compared to those treated with placebo.

### EXAMPLE 3

### Manufacture of a Stent

A stent (e.g. a Multi-Link Vision stent, Guidant Corp.; or a DRIVER stent, Medtronic Corp.) is weighed and then mounted on a rotating or other support for coating with a polymeric or other synthetic or biological carrier used as a drug reservoir. In an exemplary carrier application procedure, while the stent is rotating, a 100 μl aliquot of a solution of polylactide glycolide, 0.75 mg/ml of pimecrolimus and 0.0015 mg/ml 2,6-di-tert-butyl-4-methylphenol dissolved in a 50:50 mixture of methanol and tetrahydrofuran, is coated onto it. The coated stent is removed from the support and allowed to air-dry. After a final weighing the amount of coating on the stent is determined.

### EXAMPLE 4

### Drug Release from Polymer Coatings in Aqueous Solution

Four 2 cm pieces of stents coated as described in Example 3 above are placed into 100 ml of PBS having a pH of 7.4. Another 4 pieces from each series are placed into 100 ml of PEG/water solution (40/60 v/v, MW of PEG=400). The stent pieces are incubated at 37° in a shaker. The buffer and PEG solutions are changed daily and different assays are performed on the solution to determine the released pimecrolimus concentrations. By such method a stable pimecrolimus release from coated stents can be shown. The term “stable pimecrolimus” means that less than 10% variation of the drug release rate is observed.

### EXAMPLE 5

### Drug Release from Polymer Coatings in Plasma

Release of pimecrolimus in plasma is also studied. 1 cm pieces of a coated stent are put into 1 ml of citrated human plasma (from Helena Labs) in lyophilized form and reconstituted by adding 1 ml of sterile deionized water. Three sets of stent plasma solutions are incubated at 37° and the plasma is changed daily. Different assays are performed on the solution to determine the released pimecrolimus concentrations. By such method a stable pimecrolimus release from coated stents in plasma can be demonstrated. The term “stable pimecrolimus release” means that less than 10% variation of the drug release rate is observed.

### EXAMPLE 6

### Drug Stability in Pharmaceutically Acceptable Polymers at Body Temperature

PDGF-stimulated receptor tyrosine kinase assay can be performed on the last piece of each sample to determine the pimecrolimus activity. A similar test can be performed with free pimecrolimus. The inhibition of PDGF-stimulated receptor tyrosine kinase activity in vitro can be measured in PDGF receptor immunocomplexes of BALB/c 3T3 cells, analogously to the method described by E. Andrejauskas-Buchdunger and U. Regenass in Cancer Research 52 (1992) 5353-5358. By such approach the stability of free pimecrolimus and pimecrolimus in polymer coatings can be compared.

In Examples 1 to 6 pimecrolimus may be replaced with ABT-281, 5,6-dehydroascomycin or ASD 732 with similar results.

### Clinical Trial

The favorable effects of the anti-inflammatory ascomycin derivative pimecrolimus used according to the invention can furthermore be demonstrated in a randomized, double-blind multi-center trial for revascularization of single, primary lesions in native coronary arteries, e.g. along the following lines:

The primary endpoint is in-stent late luminal loss (difference between the minimal luminal diameter immediately after the procedure and the diameter at six months). Secondary endpoints include the percentage of in-segment stenosis (luminal diameter of stented portion plus the 5 mm proximal to and distal from the stented portion of the vessel), and the rate of repeat revascularization needed at the site of target vessel stenting. After six months, the degree of neointimal proliferation, manifested as the mean late luminal loss in the group treated with a coated stent comprising pimecrolimus versus the placebo group treated with a non-coated stent is determined, e.g. by means of a virtual, conventional catheter-based coronary angiography, and/or by means of intracoronary ultrasound.

1. A drug delivery device or system comprising:
a) a medical device adapted for local application or administration in hollow tubes; and, in conjunction therewith,
b) a therapeutic dosage of an anti-inflammatory ascomycin derivative in free form or, where such forms exist, in pharmaceutically acceptable salt form.

2. A device or system according to claim 1 wherein the anti-inflammatory ascomycin derivative is pimecrolimus in free form or, where such forms exist, in pharmaceutically acceptable salt form.

3. A device or system according to claim 1 or 2 wherein the medical device is a catheter-based delivery device or an intraluminal device adapted for local application or administration in hollow tubes.

4. A device or system according to claim 1 or 2 wherein the medical device is a catheter-based delivery device, a local injection device or system, an intraluminal or indwelling device adapted for local application or administration in hollow tubes, a stent, a coated stent, an endolumenal sleeve, a stent-graft, a controlled release matrix, a polymeric or biological endoluminal paving, or an adventitial wrap.

5. A device or system according to claim 1 or 2 wherein the anti-inflammatory ascomycin derivative is affixed to the medical device in a way allowing drug release.

6. A device or system according to claim 1 or 2 which comprises a coated stent.

7. Use of an anti-inflammatory ascomycin derivative in free form or, where such forms exist, in pharmaceutically acceptable salt form, in the preparation of a medicament for the prevention and treatment of inflammatory complications following vascular injury, such as:
prevention or treatment of vascular inflammation or smooth muscle cell proliferation and migration, or aneurysm expansion in hollow tubes, or increased extracellular matrix degradation and erosion in hollow tubes, or increased inflammatory cell infiltration, or increased cell proliferation or decreased apoptosis, or increased matrix deposition or degradation, or increased positive, aneurysmal remodeling (aneurism dilation) following device placement; or
treatment of intimal thickening or aneurysm expansion in vessel walls; or
stabilising atherosclerotic plaques, or stabilising sites of aneurysm; or
stabilising or reducing aneurysm dilation at the site of aneurysm.

8. A method of treatment of inflammatory complications following vascular injury, such as for:
preventing or treating vascular inflammation or smooth muscle cell proliferation and migration, or aneurysm expansion in hollow tubes, or increased extracellular matrix degradation and erosion in hollow tubes, or increased inflammatory cell infiltration, or increased cell proliferation or decreased apoptosis, or increased matrix deposition or degradation, or increased positive, aneurysmal remodeling (aneurism dilation) following device placement in a mammal in need thereof, comprising administration of a therapeutically effective amount of an anti-inflammatory ascomycin derivative;
treating intimal thickening or aneurysm expansion in vessel walls in a mammal in need thereof, comprising controlled delivery from a catheter-based or intraluminal medical device of a therapeutically effective amount of an anti-inflammatory ascomycin derivative;
stabilising atherosclerotic plaques or stabilising sites of aneurysm, or stabilising or reducing aneurysm dilation at the site of aneurysm in a mammal in need thereof, comprising administration of a therapeutically effective amount of an anti-inflammatory ascomycin derivative;
optionally together with one or more other active ingredients; whereby the anti-inflammatory ascomycin derivative is in free form or, where such forms exist, in pharmaceutically acceptable salt form.

9. A method according to claim 8 wherein the underlying condition beneficially affected is stenosis, restenosis, vascular inflammation, thrombosis, unstable angina, myocardial infarction, heart failure, ischaemia, sudden death, stroke and/or aneurysm rupture, and wherein the anti-inflammatory ascomycin derivative is administered from a stent or from a coating applied to a stent or is administered in conjunction with a stent.
